(19) United States
(12) Patent Application Publication
(10) Pub. No.: US 2025/0248649 A1
(43) Pub. Date: Aug. 7, 2025

(54) SYSTEMS, METHODS, AND COMPONENTS THEREOF RELATING TO RESPIRATION SEVERITY MEASUREMENT, ASSESSMENT, AND TREATMENT

(71) Applicant: Wesper Inc., New York, NY (US)

(72) Inventors: Nathan BENNETT, Brooklyn, NY (US); Walker NELSON, Brooklyn, NY (US); Mordechai PERLMAN, Cresskill, NJ (US); Chelsie ROHRSCHEIB, Howell, MI (US); Amir REUVENY, New York, NY (US)

(21) Appl. No.: 18/854,758

(22) PCT Filed: Apr. 6, 2023
(86) PCT No.: PCT/US2023/017741

§ 371 Date: Oct. 7, 2024

Related U.S. Application Data
(60) Provisional application No. 63/328,848, filed on Apr. 8, 2022.

(51) Int. Cl. A61B 5/00; A61B 5/087; A61B 5/113; A61B 5/1455; A61M 21/00

(52) U.S. Cl. CPC A61B 5/4818; A61B 5/087; A61B 5/113; A61B 5/14551; A61B 5/7246; A61M 21/00; A61M 2021/0022; A61M 2021/0027; A61M 2021/0072; A61M 2021/0083

(57) ABSTRACT

Methods and systems for assessment, treatment, and/or prevention of positional sleep therapy for sleep apnea and other disorders are disclosed. In one example, a method for determining at least one respiratory severity metric for an individual's sleep session is provided. The method may include steps of receiving a data signal indicative of respiratory air flow of the individual as a function of time; identifying a plurality of respiratory severity events from the data signal; calculating respiratory severity values for each of the plurality of identified respiratory events; grouping the plurality of respiratory events into a plurality of clusters; and calculating, for each cluster, an accumulated respiratory severity value as a clustering index.

This application claims priority to U.S. Provisional Patent Application Ser. No. 63/328,848, filed Apr. 8, 2022, the disclosure of which is hereby incorporated by reference in its entirety.

### TECHNICAL FIELD

This application relates to systems, methods, and components thereof to detect, assess, prevent, mitigate and/or treat sleep disorders, including, but not limited to, sleep apnea. The teachings of this disclosure may also be applicable to medical and wellness conditions outside the sleep disorder context, as well as to other sleep-related conditions.

### BACKGROUND

The apnea-hypopnea index (“AHI”) scale is a medically-accepted, industry-standard scale used to indicate whether an individual may exhibit breathing disturbances during sleep and sleep disorders (such as apnea or hypopnea) as well as the severity of such disorders. Apnea is sometimes defined as a reduction of airflow by at least 90% for at least 10 seconds. Hypopnea is a period of shallow breathing, which is sometimes be defined as at least a 30% reduction in nasal pressure for at least 10 seconds and coinciding with at least a 3% or 4% drop in blood oxygen saturation. An AHI number indicates the average number of times per hour that an individual has a significant, measurable episode of apnea or hypopnea. AHI is commonly determined in a sleep study in a sleep lab. AHI does not reflect the duration of each episode (i.e., beyond meeting the minimum definitional duration), the relative clustering of sleep disorder episodes, or the amplitude (e.g., reduction of airflow beyond 90%) of the of sleep disorder episodes.

While AHI may be considered a very reliable indicator of sleep disorders and their severity when AHI is less than 5 or more than 25, AHI measurements between 5 and 25 are sometimes considered less predictive and/or reliable. For example, a person with an AHI of 10 may have a severe or dangerous sleep disorder; may have a moderately harmful sleep disorder; or may just technically have a sleep disorder.

Notwithstanding that AHI measurements are the current, predominant standard of care for assessing sleep disorders, there is a need for an additional metric(s) to supplement and/or replace the AHI scale to improve diagnosis, assessment, and, in turn, treatment and mitigation of sleep disorders. This may be especially true for symptomatic patients with a low or medium AHI. There is also a need for algorithms, systems, devices, and software that may collect and/or process data from sleep study sensors or the like to automatically generate such processed metric(s) that can be readily used my sleep, medical, and/or wellness professionals, or by persons suffering from sleep disorders.

### SUMMARY

The present disclosure provides a description of algorithmic methods, apparatuses, systems, and software to address the perceived problems described above. In some embodiments, one or more monitoring devices that collect biometric data, audio data, motion data, and/or position data may be affixed to an individual; such data may be processed and evaluated to assess sleep disorders and their severity.

In some embodiments, at least one non-transitory computer readable storage medium storing a computer program is provided. When executed by a computer, for example, on a server, smart phone, PC, and/or tablet, the computer program(s) may perform the algorithm embodiments described herein, or a relevant portion thereof. It is to be understood that the descriptions herein are exemplary and explanatory only and are not restrictive of the inventive concepts disclosed.

In one example, a method for determining at least one respiratory severity metric for an individual's sleep session is provided. The method may include steps of receiving a data signal indicative of respiratory air flow of the individual as a function of time; identifying a plurality of respiratory severity events from the data signal; calculating respiratory severity values for each of the plurality of identified respiratory events; grouping the plurality of respiratory events into a plurality of clusters; and calculating, for each cluster, an accumulated respiratory severity value as a clustering index.

The method may further include designating the highest calculated clustering index, a global measure of accumulated flow reduction during the plurality of identified respiratory events, and/or a global measure of all accumulated flow reduction within identified clusters as a respiration severity metric(s).

The method may further include receiving a second data signal indicative of a position of the individual as a function of time, and recording the position of the individual for each cluster.

The step of receiving the data signal indicative of respiratory air flow may include receiving a signal from a nasal pressure sensor or an air flow sensor and/or receiving a signal from a sensor measuring relative movement of the individual's chest.

The step of identifying the plurality of respiratory severity events from the data signal may include identifying each respiratory severity event if and only if the data signal indicates that a clinical definition of apnea or hypopnea has been met and/or identifying each respiratory severity event if and only if the data signal indicates there has been a predefined reduction in air flow for at least a set period of time.

The step off calculating the respiratory severity of each of the plurality of identified respiratory events may include, for each identified respiratory event, calculating an instantaneous flow reduction signal and summing the instantaneous flow reduction signal. The step of calculating the instantaneous flow reduction signal may include calculating the instantaneous flow reduction signal as an instantaneous reduction in air flow as a relative value expressed with reference to a base level of air flow.

The step of grouping the plurality of respiratory events into clusters may comprise including, in a first cluster, a first respiratory event and all subsequent respiratory events, if any, until an air deficit signal indicates a respiratory recovery of the individual. The method may further include calculating the air deficit signal to predict the air deficit of the individual as a function of time. The step of calculating the air deficit signal may further include increasing the air deficit signal in accordance with the respiratory severity value of each identified respiratory event; and decreasing the air deficit signal during periods of time where none of the plurality of identified respiratory events are occurring. The step of decreasing the air deficit signal during periods of time where none of the plurality of identified respiratory events are occurring may include at least one of: decaying the air deficit signal linearly based on a set time window, decaying the air deficit signal linearly based on a set rate of decay, decaying the air deficit signal non-linearly based on a set time window, decaying the air deficit signal through application of a fixed length convolution mask, decaying the air deficit signal linearly based on a variable time window, decaying the air deficit signal linearly based on a variable rate of decay, and decaying the air deficit signal at a rate or function based upon the individual's personal characteristics.

The method may be performed in real time during the individual's sleep session. The method may further include generating a command to provide wake up signal if the accumulated respiratory severity for at least one cluster exceeds a threshold level and/or the second data signal indicates the individual is in a supine position.

In another embodiment, a non-transitory computer readable storage medium is provided. It may store a computer program, which when executed by a computer, perform various methods disclosed herein.

### BRIEF DESCRIPTION OF THE DRAWINGS

The accompanying drawings, which are incorporated into and constitute a part of this specification, illustrate several embodiments and aspects of the apparatuses and methods described herein and, together with the description, serve to explain the principles of the invention.

FIG. 1 is a high-level architecture diagram illustrating an exemplary respiration severity assessment system, in accordance with exemplary embodiments.

FIG. 2 is a block diagram illustrating an exemplary mobile device of FIG. 1, in accordance with exemplary embodiments.

FIG. 3 is a flow chart of an exemplary method of assessing respiratory severity, in accordance with exemplary embodiments.

FIG. 4 is a flow chart of an exemplary method of clustering respiratory severity events, in accordance with exemplary embodiments.

FIGS. 5A-5D are charts illustrating respiratory flow sensor data taken from a sleeping individual, flow amplitude data, an instantaneous respiratory severity signal, and clustered respiratory severity metrics, respectively, in accordance with exemplary embodiments.

FIGS. 6A-6C are charts illustrating respiratory flow sensor data taken from sleeping individual, an instantaneous respiratory severity signal, and clustered respiratory severity metrics, respectively, in accordance with exemplary embodiments.

FIGS. 7A-7D are charts illustrating respiratory flow sensor data taken from sleeping individual, an instantaneous respiratory severity signal, clustered respiratory severity metrics, and corresponding sleep position sensor data, respectively, in accordance with exemplary embodiments.

FIGS. 8A-8D are charts illustrating respiratory flow sensor data taken from sleeping individual, an instantaneous respiratory severity signal, clustered respiratory severity metrics, and corresponding sleep position sensor data, respectively, in accordance with exemplary embodiments.

FIGS. 9A-9C are charts illustrating respiratory flow sensor data taken from sleeping individual, an instantaneous respiratory severity signal, and clustered respiratory severity metrics, respectively, in accordance with exemplary embodiments.

FIG. 9D is a table of additional respiratory severity data derived from the flow sensor data of FIG. 9A, in accordance with exemplary embodiments.

FIGS. 10A and 10B are charts validating that respiratory severity metrics derived from sensor data in accordance with exemplary embodiments are correlated with hypoxic burden.

FIG. 11 is a chart illustrating the utilizing of respiratory severity metrics derived from sensor data in accordance with exemplary embodiments provides an advantage over using AHI alone.

### DETAILED DESCRIPTION

### Respiratory Severity Assessment System 100

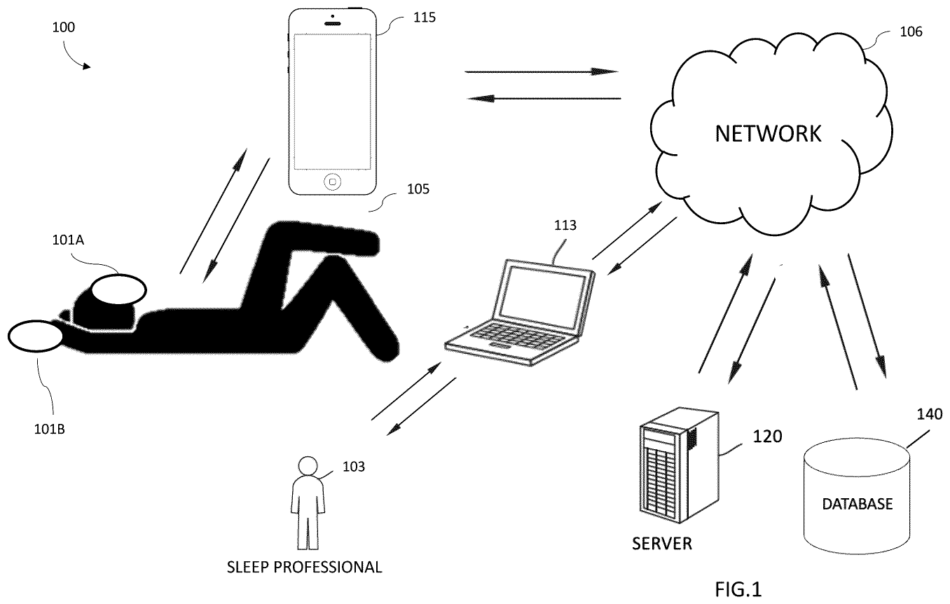

FIG. 1 illustrates an embodiment of respiratory severity assessment system 100. While the disclosed methods and systems are explained herein in the context of detecting, assessing, preventing, and mitigating sleep disorders, they are not so limited. That is, the disclosed methods and systems may be embodied in other contexts and technologies, as would be understood by a person of ordinary skill in the art.

System 100 may include one or more person 105, one or more mobile or computing devices 115, and one or more monitoring devices 101, including, but not limited, to air flow measuring sensor 101A, blood oxygenation sensor 101B, and/or air pressure sensor (not shown).

Monitoring devices 101 may be configured to attach to a person 105 and obtain breathing flow, blood oxygen saturation, breathing pressure, positional, movement, biometric, and/or other relevant sensor data therefrom. In some embodiments (not shown), monitoring devices 101 may be configured to provide tactile, audible, or electric shock feedback to person 105 to, for example, fully or partially wake up person 105 if a sleep disorder episode(s) or sleep disorder episode(s) of a particular severity is detected (and such monitoring devices 101 may or may not also collect data). Monitoring devices 101 may be configured to communicate with one or more mobile devices 115 via Bluetooth and/or another wireless or wired communication protocol.

One or more persons 105 may use mobile device 115 running application software and/or a web interface to interact with other components of system 100. In preferred embodiments, mobile device 115 may be a smart phone running, for example, Apple® OS, Android®, or the like. Mobile device 115, discussed in more detail below, however, is not so limited: It may be any type of mobile computing device suitable for performing the functions and algorithms disclosed herein such as a smart watch, laptop computer, notebook computer, tablet computer, etc. In alternative embodiments, it may be a computing device that is not mobile. Mobile device 115 may be connected to a network 106, and may connect to server 120 therethrough.

It is contemplated that system 100 may concurrently provide support to multiple persons 105 by interfacing with multiple mobile devices 115 and/or their respective connected monitoring device(s) 101, respectively.

Network 106 may be any type of network suitable for performing the functions disclosed herein as will be apparent to persons having skill in the relevant art; it may include one or more communications technologies, including, but not limited to 3G, LTE, 4G, 5G, WiMax, the Internet, etc.

System 100 may include one or more sleep, wellness, and/or medical professionals 103 who may conduct a sleep study on persons 105 and/or otherwise manage sleep issues for persons 105. Professionals 103 may use computing device 113 running application software and/or a web interface to interact with other components and users in system 100. Computing device 113 may be any type of computing device suitable for performing the functions and algorithms disclosed herein such a desktop computer, laptop computer, notebook computer, tablet computer, smartphone, and/or alternatively may be substantially the same as mobile device 115. In some embodiments, computing device 113 may be additionally utilized to perform conventional sleep studies via, for example, air flow measuring sensor 101A, blood oxygenation sensor 101B, and/or air pressure sensor (not shown). Computing device 113 may be connected to network 106, and may connect to server 120 therethrough. It is contemplated that system 100 may concurrently provide service to multiple sleep professionals 103 concurrently.

System 100 may include one or more system administrator 107 (not shown). Admin 107 may use computing device 117 (not shown) to access, control, program, enter data in, receive data from, and update server 120 and other system 100 components. Admin 107 is not limited to an individual with an administrator title, but may include any person or group of people using computing device 117 to manage or otherwise directly alter the software structures, functions, or data of server 120. Computing device 117, may be any type of computing device suitable for performing the functions and algorithms disclosed herein such a desktop computer, laptop computer, notebook computer, tablet computer, smartphone, etc. Computing device 117 may be connected to network 106, and may connect to server 120 there through. Computing device 117 may be utilized to upload software, software updates, and/or software patches to network 106 for distribution and installation on devices 113/115.

Server 120 may substantially comprise the back-end of system 100 and may include and/or access database 140. As would be appreciated by a person of skill in the art, server 120 may be cloud-based, may comprise one or more server 120 installations, and/or may be distributed. Database 140 may be a cloud-based database (as shown in FIG. 1) and/or may be co-located with processing elements of server 120.

Server 120 may enable or facilitate data storage and data transmission relating to persons 105; and/or downloading and updating application software; it may support one or more web interfaces. Server 120 may additionally comprise a plurality of functional software blocks, to accomplish for example, data intake, anonymization, aggregation, and analysis. In some embodiments, certain functional software blocks of server 120 may interface with one or more third party services directly and/or over network 106 via, for example, API calls or the like to obtain or report sleep data or other information to medical providers, facilitate financial transactions, and/or the like.

It is specifically contemplated that algorithmic data processing disclosed herein may be run on one or more elements of system 100. For example, in certain embodiments of system 100, all or most data processing may occur in the cloud—e.g., run by Server(s) 120. However, it is contemplated that in other embodiments, all or most of such algorithmic processing data may occur on a mobile and/or computing devices 113/115.

### Mobile/Computing Devices 113/115

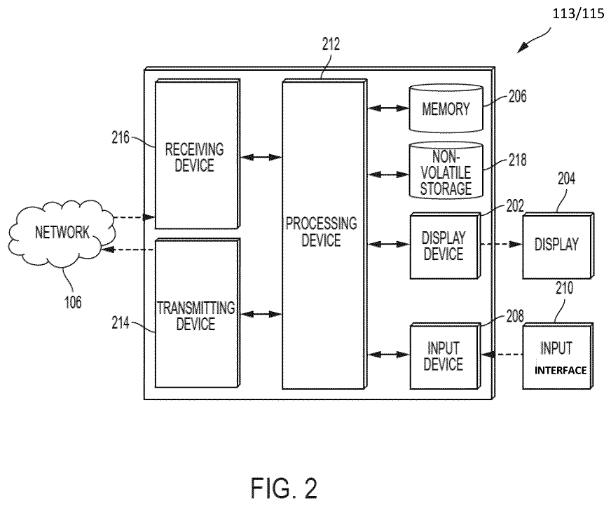

FIG. 2 illustrates an embodiment of mobile and/or computing device 113/115 of system 100. It will be apparent to persons having skill in the relevant art that the embodiment of the device 113/115 illustrated in FIG. 2 is provided as illustration only and may not be exhaustive to all possible configurations of a device 113/115 suitable for performing the functions as discussed herein. Device 113/115 may include a display device 202. The display device 202 may be configured to communicate and/or interface with a display 204 to display data to a user 103/105. The display 204 may be any type of display suitable for performing the functions disclosed herein, such as a liquid crystal display, light-emitting diode display, OLED display, touch screen display, capacitive touch display, etc. The display device 202 may be configured to transmit data to the display that is stored in a memory 206 of the device 113/115.

Device 113/115 may include non-volatile storage 218, which can store software and data that would be apparent to persons having skill in the relevant art.

The display device 202 may be configured to display various interfaces and appropriate data to the relevant user 103/105. The display device 202 may also display a cursor position, which may allow user 103/105 to select an option or a variable, use a dropdown menu, indicate a point of input for text or commands input by the user 103/105, and/or facilitate user communication in another fashion known to persons of skill in the art.

Device 113/115 may receive input from user 103/105 via an input device 208. User 103/105 may communicate with the input device 208 via an input interface 210 that is connected to or otherwise in communication with the input device 208. The input interface 210 may be any type of input suitable for performing the functions disclosed herein, such as a keyboard, mouse, touch screen, click wheel, scroll wheel, trackball, touch bad, input pad, microphone, camera, etc. In some embodiments, the input interface 210 and the display 204 may be combined, such as in a capacitive touch display. In some instances, the display 204 and/or the input interface 210 may be included in the device 113/115. In other instances, the display 204 and/or the input interface 210 may be external to the device 113/115.

Device 113/115 may further include a processing device 212. The processing device 212 may be a central processing unit (CPU) or other processor or set of processors suitable for performing the functions disclosed herein as will be apparent to persons having skill in the relevant art. The processing device 212 may receive data associated with input by a user, such as via the input device 208. The processing device 212 may also be configured to read data and software stored in non-volatile storage 218 and memory 206; write data and software stored in non-volatile storage 218 and memory 206; execute program code stored in the memory 206 or non-volatile storage 218, such as embodiments of the algorithms disclosed herein; communicate to other system 100 components on network 106 via receiving device 216 and transmitting device 214; and transmit data to the display device 202 for display to the user 103/105 via the display 204. The processing device 212 may be further configured to execute embodiments of the algorithms disclosed herein, as discussed in more detail below. Additional functions performed by the processing device 212 will be apparent to persons having skill in the relevant art and may also be discussed herein.

The memory 206 may store data suitable for performing the functions disclosed herein. Some or all of the data and software stored within non-volatile storage 218 may be copied to memory 206 to support the processing functions of processing device 212.

Device 113/115 may also include a transmitting device 214. The transmitting device 214 may be configured to transmit data over the network 16 via one or more suitable network protocols. Device 113/115 may also include a receiving device 216. The receiving device 216 may be configured to receive data over the network 110 via one or more suitable network protocols.

It is also specifically contemplated that transmitting and receiving devices 214/216 or at least mobile device 115 are configured to communicate via Bluetooth protocol or the like to exchange data and/or commands with monitoring device 101.

### Monitoring Devices 101

Monitoring devices 101 may contain various sensors including, but not limited to, air flow sensors (101A); Sp02 sensors (101B); pressure sensors; position sensors; motion sensors; pulse sensors; proximity sensors; microphones; bio-impedance sensors; electro-optical sensors comprising, for example, light source and photodetector pairs; electro-mechanical sensors comprising, for example, flexible, conductive sheets; and/or other sensors known in the art to collect other relevant biometric data.

While it is contemplated that respiration severity metrics may be generate using air flow measuring sensor 101A as the only sensor or monitoring device 101, this disclosure is not so limited. Utilization of Sp02 sensor 101B, a pressure sensor, and/or other sensors and corresponding monitoring devices 101 are specifically contemplated.

With reference to U.S. Pat. Nos. 10,531,832 and 10,531,833, and U.S. Patent Pub Nos. 2020/0155071 and 2020/0107782—which are hereby incorporated herein in their entireties, some embodiments of monitoring device 101 may be a patch. In such patch embodiments, monitoring devices 101 may be affixed to an individual's skin and/or may serve to detect breathing-related bodily movements.

In some embodiments, one or more stimulation components may be integrated into monitoring device(s) 101. Such stimulation components may provide electrical stimulation, for example, akin to a TENS unit; vibration or other tactile stimulation, for example, via a small electric motor; auditory stimulation, for example, via a speaker; and/or the like.

A person 105 employing system 100 may utilize one or more monitoring devices 101 concurrently in some embodiments. Each monitoring device 101 being utilized for a single person 105 may transmit and receive data with a paired mobile/computing device 115/113 via its communications circuitry. Such data may include processed or unprocessed sensor data from monitoring device 101; battery or power consumption data from monitoring device 101; stimulation commands from mobile/computing device 115; various signal receipt confirmations; and/or the like.

During use, one or more monitoring devices 101 may be attached to an individual 105. The simultaneous use of multiple monitoring devices 101 may serve to provide a more robust or reliable array of sensor data on a person 105, thereby enabling more detailed or reliable sleep disorder severity or other biometric analysis.

Furthermore, it is contemplated that different types of monitoring devices 101 with varied sensor and/or stimulation component sets may be provided. This may enable the most appropriate sensor(s) and/or stimulation component(s) to easily be selected and used in view of the preferences and needs of each person 105 and their particular circumstances. Moreover, the inclusion of an excessive amount of sensor(s) and/or stimulation component(s) may be undesirable in view of the corresponding manufacturing cost, power consumption, size, weight, data processing limitations, sensor interference, and/or the like.

In certain embodiments, for example where multiple monitoring devices 101 are utilized, one or more monitoring devices 101 may contain one or more stimulation components to the exclusion of sensors. In such embodiments, “monitoring device” may be a misnomer and monitoring device 101 may be considered a dedicated wake device.

In alternative embodiments, one or more monitoring devices 101 may contain sufficient processing circuitry and software to enable data-processing and decision-making without being connected to, communicating with, and/or relying upon mobile/computing device 113/115 or another external device. In such embodiments, monitoring device(s) 101 may contain additional circuitry and/or software components discussed above with respect to mobile/computing device 113/115. It is contemplated that in some embodiments where multiple monitoring devices 101 are utilized, one monitoring device 101 may contain additional processing and execution functionality and serve as the “master”; additional monitoring device(s) 101 may be considered “slave(s).” The slave monitoring devices 101 may communicate with the master, with the master collecting data from and issuing commands to the slave(s).

### Respiration Severity Assessment Method 300

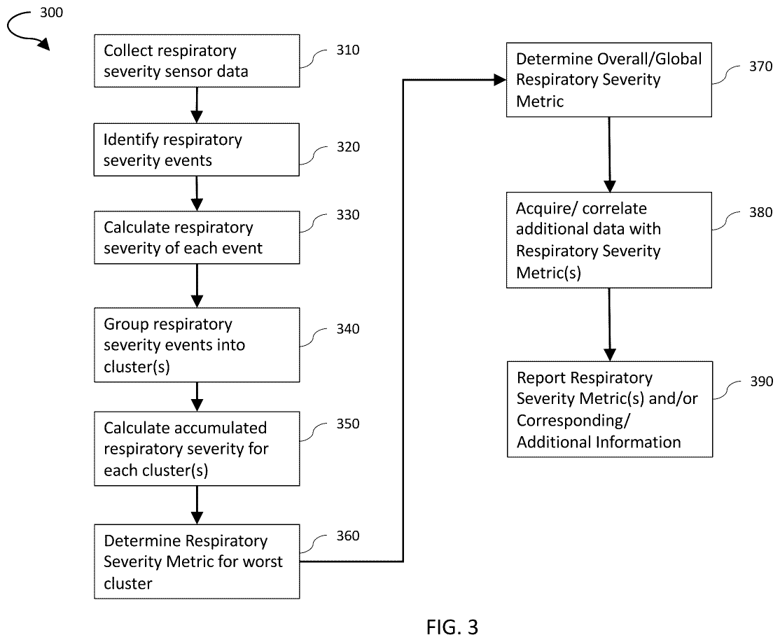

An exemplary embodiment of Respiration Severity Assessment Method 300 is depicted in FIG. 3.

As in step 310, respiration severity sensor data may be collected. In certain exemplary embodiments, such data may consist of, comprise, or substantially comprise, at least on directly or indirectly measured breathing air flow data. This disclosure specifically contemplates additionally or alternatively utilizing respiration severity sensor data that may initially comprise measures of chest/body movement, pressure measurement (e.g., via directly measure nasal pressure), and/or or blood-oxygen measurements. In some embodiments, respiratory air flow and/or air pressure data may be indirectly captured via other sensor measurements. For example, with reference to U.S. Pat. No. 10,531,833, movement of user 105's chest and/or stomach area may be captured via movement sensor(s) 101 to derive respiratory effort data: From such movement sensor/respiratory effort data, air flow and/or air pressure data may be extrapolated, estimated, and/or indirectly obtained. It is contemplated that in some embodiments, multiple input signals (e.g., pressure, flow, respiratory effort, Sp02) maybe utilized for multidimensional sleep severity analysis. For example, it is contemplated that respiratory effort data in combination with, e.g., directly measured flow or pressure data, may be utilized to characterize respiratory events as either obstructive or central.

In certain exemplary embodiments, which are primarily discussed below with respect to method 300 for ease of explanation, respiration severity sensor data for a complete sleep session may be fully collected before completion of step 310. For example, breathing air flow data may be collected for a complete period of sleep in a sleep study.

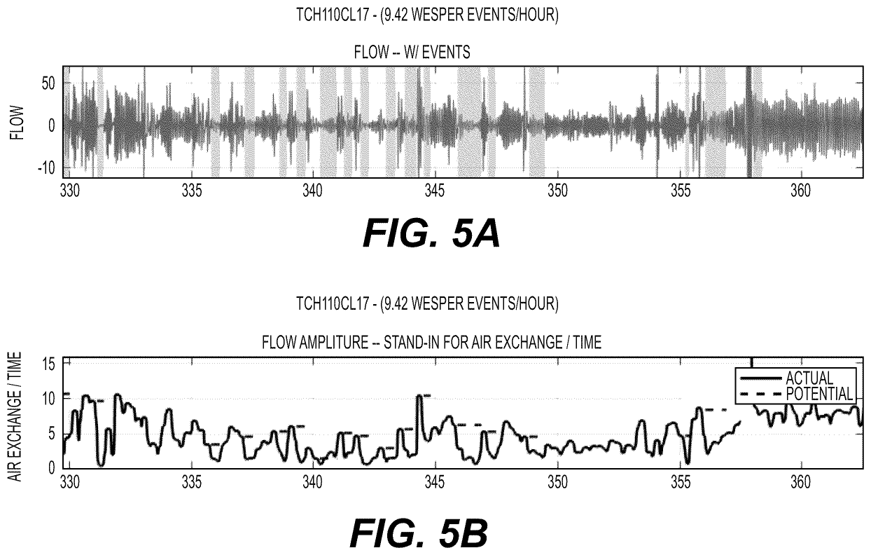

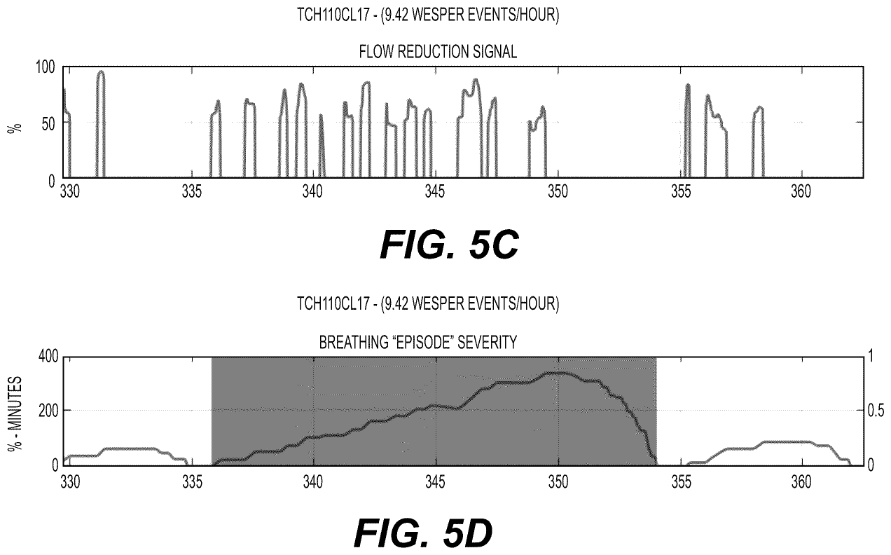

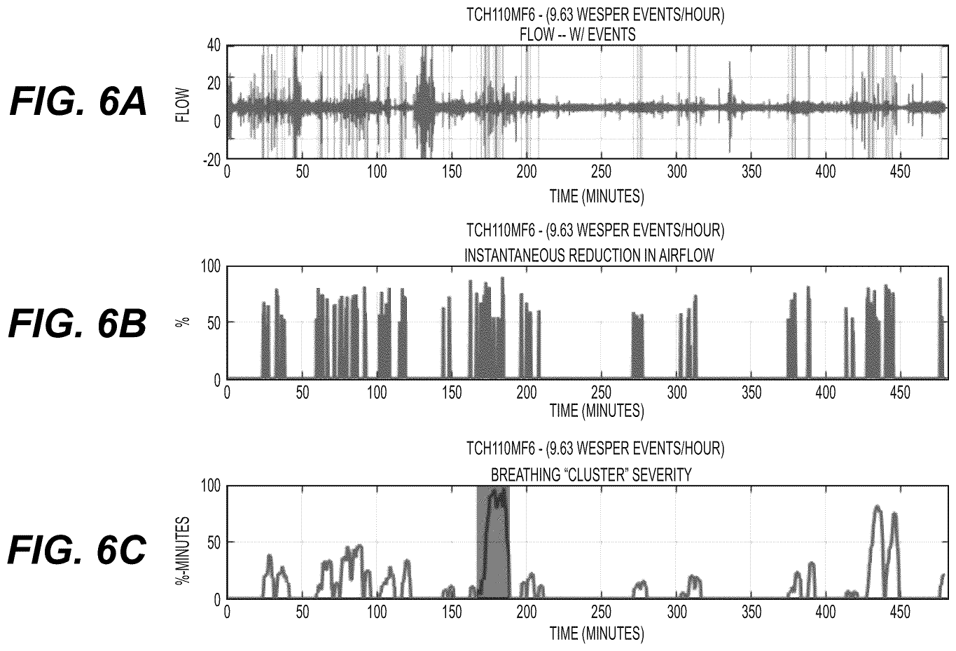

Consistent with step 310, FIGS. 5A, 6A, 7A, 8A, and 9A depict sets of air flow sensor data taken during actual sleep studies of individuals. The unit of measure for air flow in these figures are microvolts, which is uncalibrated sensor data. However, because relevant air flow metrics may be calculated and utilized as a percentage decrease in flow and/or another measure of relative amplitude change, the unit may not be of importance in certain contemplated embodiments. It is specifically contemplated that, in some embodiments air flow data may be indirectly derived from movement sensor data, pressure sensors data, and/or the like. Specifically, in certain preferred embodiments, air flow data may be indirectly derived from movement sensor data collected from movement sensing patch(es) affixed to person 105's chest and/or stomach area.

FIG. 5B depicts the flow sensor data of FIG. 5A processed in measurements of air exchange over time. The flow data may, for example, be converted into air exchange data by taking the absolute value of the flow sensor data. In some embodiments, the data may further be processed to smooth the signal. It is noted that air flow data commonly utilized for generating an AHI value may, in some exemplary embodiments, be sufficient by itself. Method 300 may proceed to step 320.

As in step 320, respiratory disorder events may be identified by analysis of the respiration severity sensor data. In some embodiments, an event may be identified when the sensor data indicates that a clinical definition of apnea has been met—e.g., at least a 90% drop in flow for at least 10 second. In other embodiments, an event may be identified when the sensor data indicates that a clinical definition of apnea, hypopnea, and/or another sleep disorder event has been met. In yet another embodiment, an event may be identified by detecting a substantial reduction in airflow for a period of time, for example a reduction of at least 50% for at least 10 seconds. Additionally or alternatively, respiratory disorder events may be identified or confirmed by utilizing resulting data from a completed, manually scored sleep study.

With reference to the sleep study data of FIGS. 5A, 6A, 7A, 8A, and 9A, respectively, each identified event is illustrated via a vertical shaded area (some of which may appear as shaded lines due to their brevity). In FIG. 5B, each identified event is illustrated via a horizontal line that represents the length of the event. Method 300 may proceed to step 330.

As in step 330, a continuous respiratory severity may be derived. With reference to FIGS. 5C, 6B, 7B, 8B, and 9B, a flow reduction signal may be calculated for each identified event. As depicted, the flow reduction signal may be calculated as an instantaneous reduction in airflow, for example expressed as a percentage of a base level of airflow.

In preferred embodiments, the base level of airflow may be defined by the airflow immediately preceding the onset of the event; that is, the flow reduction signal may comprise an expression of reduction from normal “local flow.” For example, with reference to FIG. 5B, the height of the horizontal line represents the baseline from which reduction is calculated. In alternative embodiments, the base level of airflow may be set based on global/absolute air flow for user 105, for example, during the current sleep session, over a longer period of time, or for some combination thereof. In yet other embodiments, the base level of airflow may be set based on combination of global/absolute air flow and local flow. Additionally or alternatively, the global/absolute air flow may be sleep position dependent in some embodiments. For example, a position-dependent global/absolute air flow used as a baseline when a user is in a supine position may be set based on a global/absolute air flow subset corresponding to the supine position.

In exemplary embodiments, the flow reduction signal (or the like) for each event may be utilized to derive a measure of severity for such event, for example, by taking the integral of or otherwise summing the area above and/or underneath the flow reduction signal for each such event. The event severity measure may be utilized to calculate an air deficit that the event has caused. Other statistical forms of measurement representing such severity, including mean and mode, are also contemplated. Method 300 may proceed to step 340.

As in step 340, identified events may be grouped into clusters. Each cluster of event(s) may correspond to a single severity episode. It takes a period of adequate respiration for an individual to recover from a respiratory severity event; in many cases, an individual might not recover from one event before another event begins. To wit, when events are temporally clustered together, the air deficit from which an individual suffers from may be understood to accumulate. Accordingly, each severity episode may cover a period of time wherein an individual is assessed to have been unable to recover from an immediately prior set of one or more respiratory events. Each event within an episode may be considered part of a cluster. Each episode may be understood to begin when the first event of the cluster begins and end when recovery from all events in the cluster has occurred.

Method 400, discussed below, is an exemplary embodiment of step 340, but this disclosure is not so limited. Consistent with method 400, FIGS. 5D, 6C, 7C, 8C, and 9C, depict breathing cluster severity signals derived by accumulating the flow reduction signals of corresponding events in FIGS. 5C, 6B, 7B, 8B, and 9B, respectively, and modeling respiratory recovery during the periods between identified events.

With reference to FIGS. 5D, 6C, and 9C, each cluster is observable as non-zero waveforms that corresponds to the vertically-aligned events in FIGS. 5C, 6B, and 9B, respectively.

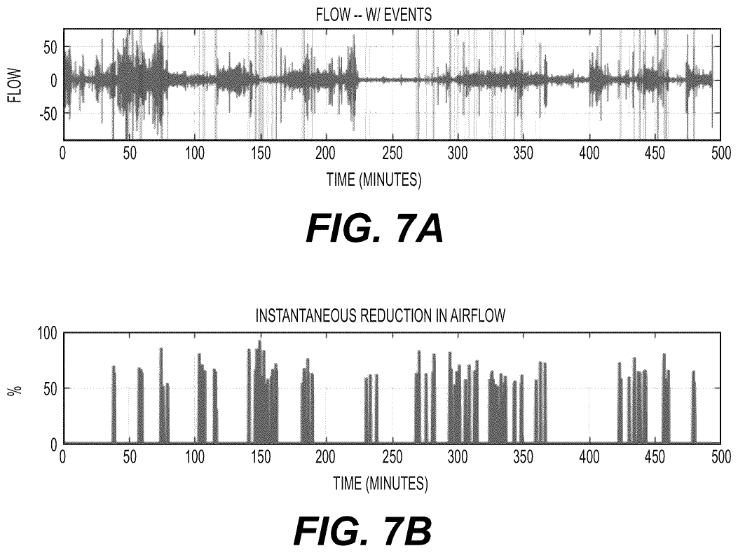

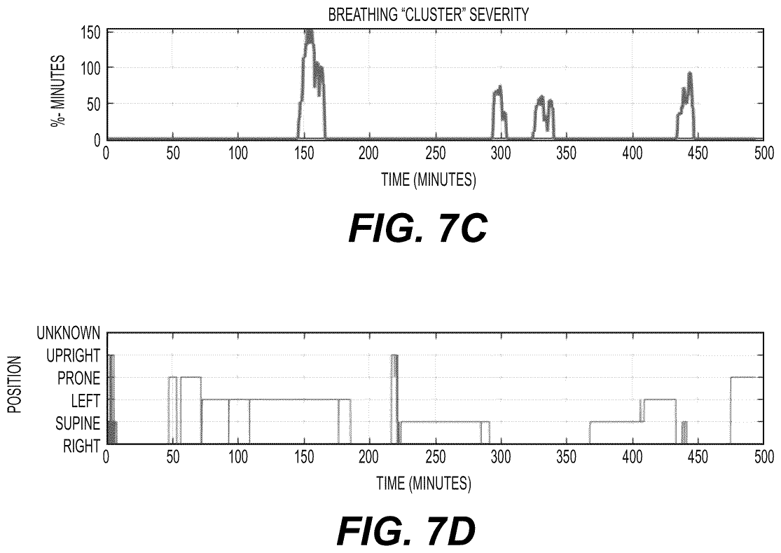

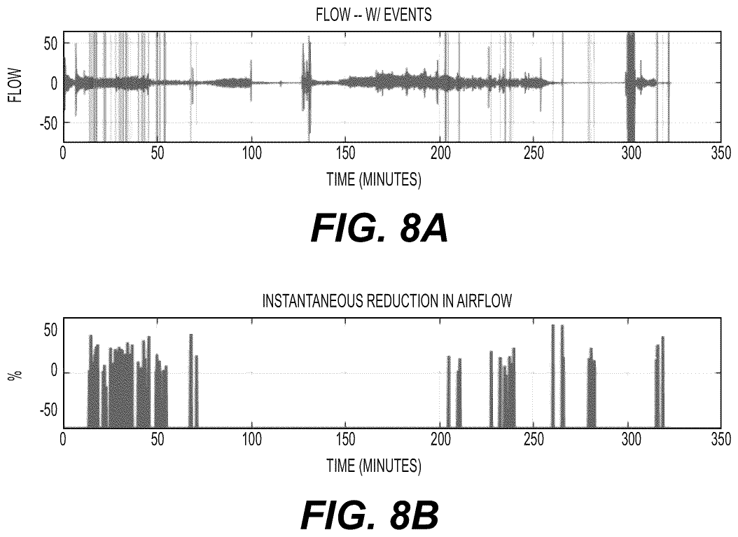

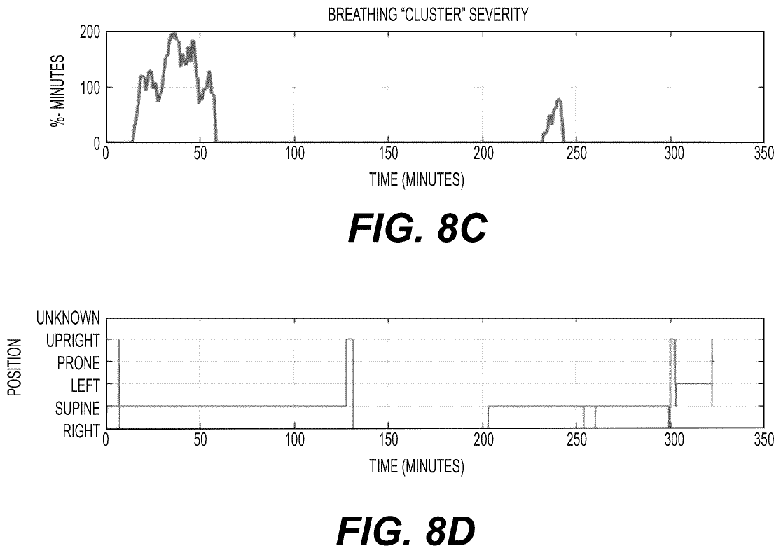

In alternative embodiments, a threshold number of consecutive severity events without full recovery must occur before a “cluster” may be identified. For example, with reference to FIGS. 7C and 8C, the cluster defining threshold utilized was five (5) events. As may be observed by comparing FIGS. 7A and 7B with 7C, respiratory events and a non-zero flow reduction signal is observable between (approximately) 25-125 minutes, 175-280 minutes, 350-375 minutes, and 450-500 minutes; however, FIG. 7C shows no clusters in those periods. As may be observed by comparing FIGS. 8A and 8B with 8C, respiratory events and a non-zero flow reduction signal is observable between (approximately) 60-75 minutes, 20-225 minutes, and 250-325 minutes; however, FIG. 8C shows no clusters in those periods. Other severity event count thresholds, for example between 3 and 7, or more broadly between 2 and 10 are additionally contemplated.

Method 300 may proceed to step 350.

As in step 350, one or more respiratory severity metrics for of each cluster or episode may be calculated. Such respiratory severity measure may be referred to as a clustering index. With reference to FIGS. 5D, 6C, 7C, 8C, and 9C, the clustering index for each identified cluster/episode may be derived by accumulating the corresponding breathing episode severity signal.

In exemplary embodiments, the clustering index may be taken as a measure of severity for such cluster/episode. The accumulated event severity measure may be utilized to calculate an air deficit that the cluster/episode has caused. Method 300 may proceed to step 360.

As in step 360, the worst (e.g., largest) clustering index may be determined. In exemplary embodiments, the episode with the largest clustering index may be identified as the worst episode, and its clustering index may be determined to be the worst clustering index. With reference to FIGS. 5D, 6C, and 9C, respectively, the worst episode is illustrated via a vertical shaded area. Method 300 may proceed to step 370.

As in step 370, one or more global respiratory severity metrics may be determined. In certain exemplary embodiments, the global respiratory severity metric may comprise a measure of all accumulated flow reduction signals of all events, for example, as taken in step 330. Such measure may be or include a sum, mean, median, max, or the like. In alternative embodiments, global respiratory severity metric may comprise the sum of all clustering indexes, for example as taken in step 350. If as discussed above with reference to FIGS. 7C and 8C in step 340, a severity event cluster defining threshold is utilized, a global respiratory severity metric derived from the cluster signal may be lower. Method 300 may proceed to step 380.

As in step 380, additional severity metric data and correlations may be derived.

For example, with reference to FIG. 7D, sensor data indicative of sleeping position is shown. By comparing FIGS. 7C and 7D is may readily be observed that the worst episode occurred when the sleeper was in the “left” position, and that all clusters coincided with the sleeper being in the “left” or “right” position. Further, by correlating derived respiration severity data with sleep position data, respiratory severity assessments for each sleep position may be derived. In this example, it has been assessed, based on respiratory severity metrics and AHI, that individual's respiratory condition in the supine position was “good” and her respiratory condition in the “left” position was “poor”; based on AHI alone, that individual's respiratory condition in both the supine and “left” positions was deemed “fair.”

Similarly comparing FIGS. 8C and 8D is may readily be observed that all episodes occurred when the sleeper was in the “supine” position. In this example, it has been assessed, based on respiratory severity metrics and AHI, that the individual's respiratory condition in the supine position was “poor”; based on AHI alone, that individual's respiratory condition was deemed “fair.”

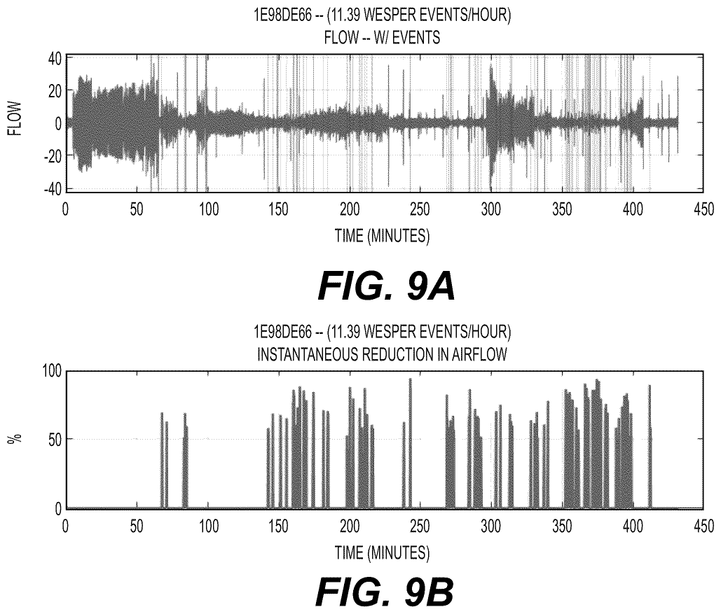

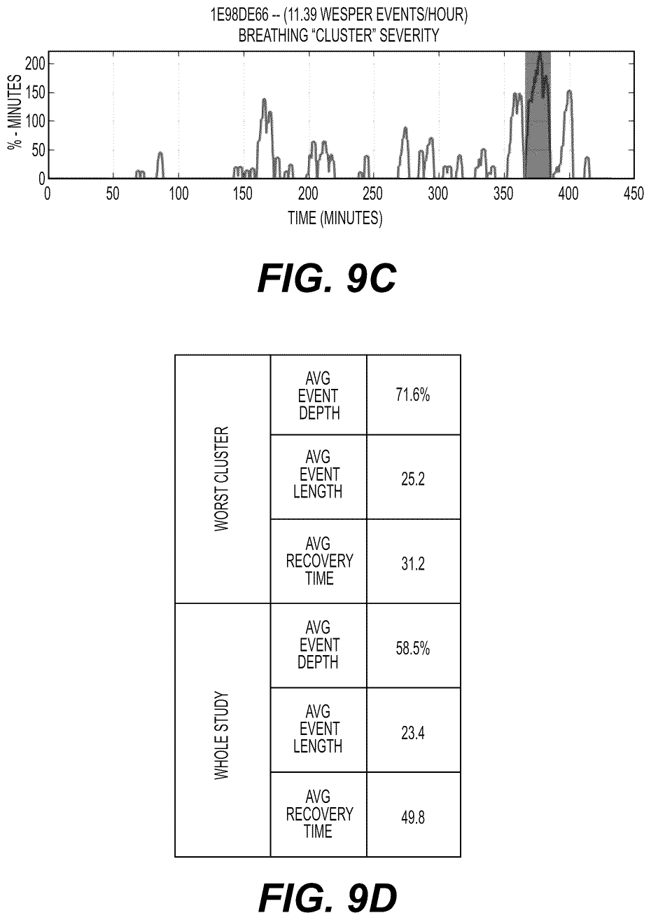

As another example, with reference to FIG. 9D, additional statistical data relating to the worst cluster/episode, events within full assessed time period, and/or clusters within the full assessed time period (not shown) may be derived. The data in FIG. 9D may be considered summary statistics of respiratory event severities. Other statistics may relate to the number of events in each cluster, the severity of the worst event globally, the severity of the worst event in one or more clusters, the length of events, and/or the like. Method 300 may proceed to step 390.

As in step 390, respiratory severity metrics and related information may be reported to subject persons 105, sleep professionals 103, appropriate medical/wellness databases 140, and or the like. The reporting may be accomplished via display on a screen, via email or another electronic message, and or the like. In some embodiments, reporting may include charts similar to those of FIGS. 5A, 5B, 5C, 5D, 6A, 6B, 6C, 7A, 7B, 7C, 7D, 8A, 8B, 8C, 8D, 9A, 9B, 9C, and/or the like. In other embodiments, summary statistics may be provided, for example, similar to FIG. 9D or the like. In other examples, summary prose, illustrative figures, or video, for example summarizing results and/or suggesting changes to sleep modes may be automatically generated and provided. Method 300 may be completed.

In alternative embodiments of respiration severity assessment method 300, for example, when used by a sleeper in the home setting, some of the aforementioned steps may occur effectively simultaneously and/or continuously. In one example, events and their severities may be quantified in real time (e.g., steps 320, 330) as sensor data is captured (e.g., step 310); cluster/episodes and their severities may be quantified in real time (e.g., steps 340, 350); interim sleep metrics may be created and updated in real time (e.g., akin to steps 360, 370); and/or data correlation and analysis may occur in real time (e.g., step 380). In this manner, system 100 may be configured to fully wake-up or partially wake-up (e.g., to prompt a change in sleep position) a person 105 based on continually updated respiration severity metrics or the like. For example, system 100 may be configured to wake-up person 105 if the current clustering index exceeds a certain threshold value. In another example, system 100 may be configured to prompt a change in sleep position if the current clustering index exceeds a certain threshold and person 105 is in a disfavored sleep position (e.g., supine). This disclosure contemplates many similar protocols that may be semi-automatically programmed/selected based on each person 105's sleep disorders and individualized treatment plans.

### Respiration Event Clustering Method 400

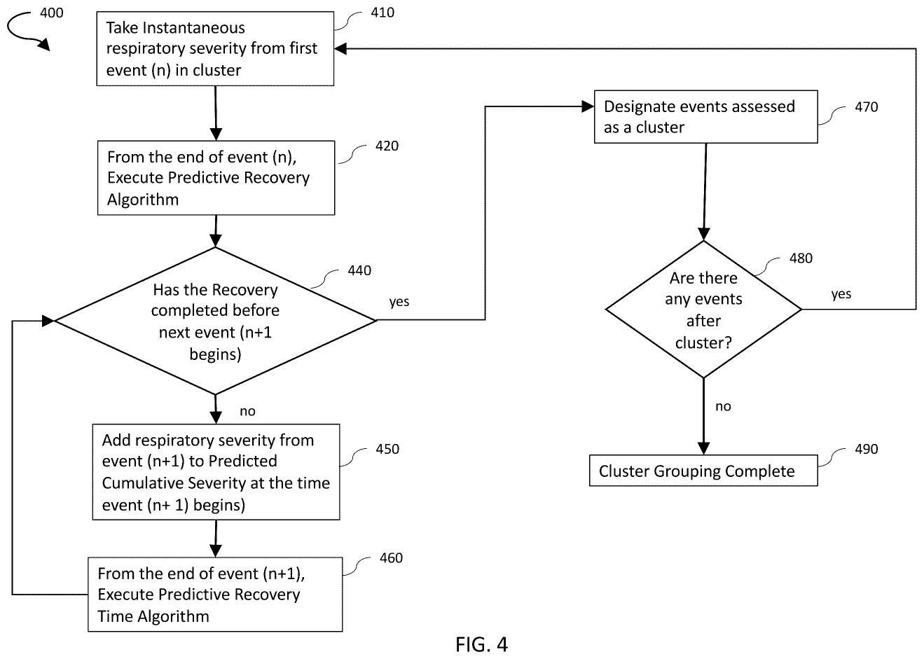

An exemplary embodiment of Respiration Event Clustering Method 400 is depicted in FIG. 4.

As in step 410, respiratory data from a first event may be taken (e.g., determined in step 330 or the like). Method 400 may proceed to step 420.

As in step 420, a predictive recovery algorithm may be executed starting at the time the event concludes and a period of “normal” respiration resumes. The predictive recovery algorithm takes the current measure of estimated air deficiency, for example as expressed in %-minutes, and reduces this deficiency over time to simulate person 105's recovery of the air deficiency.

In one embodiment, the predictive recovery algorithm may operate by simulating a linear recovery over a fixed window of time. In this embodiment, severity may decay linearly from the maximum to 0 over a fixed period of time. Here, the length of the time window is a parameter that may be set universally for all users 105; that may be set semi-universally based on a user 105's personal characteristics (e.g., age, sex, BMI, weight, fat %, sleep severity diagnosis, etc.); and/or specifically derived for a particular user 105 by, for example, assessing recovery time during a sleep study by monitoring Sp02 sensor data or the like.

In a second embodiment, the predictive recovery algorithm may operate by simulating a linear recovery with a fixed rate of decay. In contrast with the first embodiment, in this embodiment, the rate of decay (linear slope) may be fixed instead of the time to reach 0. In this embodiment, larger accumulations of severity would take a longer time to decay back to 0. Here, the rate of decay is a parameter that may be set universally, semi-universally, or via testing of user 105 as discussed above.

In a third embodiment, the predictive recovery algorithm may operate to simulate nonlinear recovery over a fixed window of time. In this embodiment, severity may decay according to some other function of time, to always reach 0 over a fixed period of time. The parameters of fixed period of time and the specific decay function used (or fixed constants therein) may be set universally, semi-universally, and/or via testing of user 105 as discussed above.

In a fourth embodiment, the predictive recovery algorithm may operate to simulate recovery according to a fixed length convolution mask. In this embodiment, severity would decay according to the coefficients of a fixed length convolution mask. This is similar to the third embodiment above, but would not necessarily be bound to be a continuous function with an analytical expression. Here, the parameters of fixed period of time and the mask coefficients may be set universally, semi-universally, and/or via testing of user 105 as discussed above.

In a fifth embodiment, the predictive recovery algorithm may operate to simulate linear recovery over a variable sized window. In this embodiment severity may decay linearly to 0 over a period of time which could vary cluster by cluster (or event by event) as a function of one or more cluster (or event) properties. It is contemplated that such properties may include, but are not limited to total accumulated severity, total length of cluster, mean event severity, number of events in cluster, and/or the like. The parameters may include which properties to use, the specific function, and/or parameters of the chosen function (e.g. coefficients, exponents, etc.); such parameters may be selected and/or set universally, semi-universally, and/or via testing of user 105 as discussed above.

In a sixth embodiment, the predictive recovery algorithm may operate to simulate linear recovery with a variable rate of decay. In such embodiment, the rate of decay (e.g., linear slope) may depend on one or more cluster or event properties similar to the fifth embodiment.

In a seventh embodiment, the predictive recovery algorithm may operate to simulate nonlinear recovery over a variable window. In such embodiment, severity may decay according to some function of time such that the severity reaches 0 over a period of time. The period of time may depend on one or more cluster (or event) properties.

In an eighth embodiment, the predictive recovery algorithm may operate to simulate recovery according to a convolution mask whose length and/or coefficients depend on one or more cluster (or event) properties.

In a ninth embodiment, the predictive recovery algorithm may employ a complex physiological model. In such embodiment, recovery may be forecast in accordance with a more sophisticated mathematical model of the physiological dynamics at play. Such model may take one or more cluster (or event) properties into account, and may or may not include more patient-specific parameters (gender, weight, body measurements, smoking status, etc.). It is contemplated that, in some embodiments, the physiological model could be embodied as a complicated function or the solution to a differential equation.

Other embodiments for the predictive recovery algorithm, including combinations of the above-described embodiments, are also contemplated. It should be noted that the settable algorithm parameters may saturate under some conditions, meaning that they only have a changing effect up to certain input value(s). For example, if a cluster property used to determine the length of a window is the number of events in a cluster, it may be desirable that the window length increases with the number of events only up to a certain point; Beyond that number, an increase in the number of events would not cause a commensurate change in window length.

Method 400 may proceed to step 440.

As in step 440, it may be assessed whether recovery has been completed (or, in alternative embodiments, substantially completed) before the next event commences. If the air deficiency has been eliminated before the next event begins, method 400 may proceed to 470 because the cluster/episode may be considered completed. If, however, the next event commences while the air deficiency still exists, method 400 may proceed to step 450 as the cluster/episode may be considered incomplete/ongoing.

As in step 450, the respiratory severity from the next event (e.g., determined in step 330 or the like) may be combined to the current measure of estimated air deficiency. It may be noted that step 450 may comprise, but is not limited to the mathematical summing of estimated severity; for example, the use of convolution with a mask that places more emphasis on more recent events is also contemplated. Method 400 may proceed to step 460.

As in step 460, the predictive recovery algorithm may be executed starting at the time the event (e.g., of step 450) concludes and a period of “normal” respiration resumes. Method 400 may return to step 440 to determine whether the cluster/episode may be considered completed.

As in step 470, the prior event(s) since the start of method 400 or the completion of the prior cluster may be assessed to be a complete cluster. However, in embodiments where a threshold number of events are required as part of a cluster definition (as discussed above with reference to FIGS. 7C and 8C), compliance with the threshold requirement may be assessed in order to designate whether the events assessed comprise a cluster. Method 400 may proceed to step 480.

As in step 480, it may be assessed whether any event exists after the just-deemed-completed cluster. If there are more events, method 400 may return to step 410 to evaluate the next event. If, however, there are no more events, it may be determined that the cluster grouping process is complete, as in step 490.

### Validation of Respiration Severity Metrics

The inventors have validated the above-discussed respiratory severity metrics as reliable indicators of sleep disorders and severity thereof.

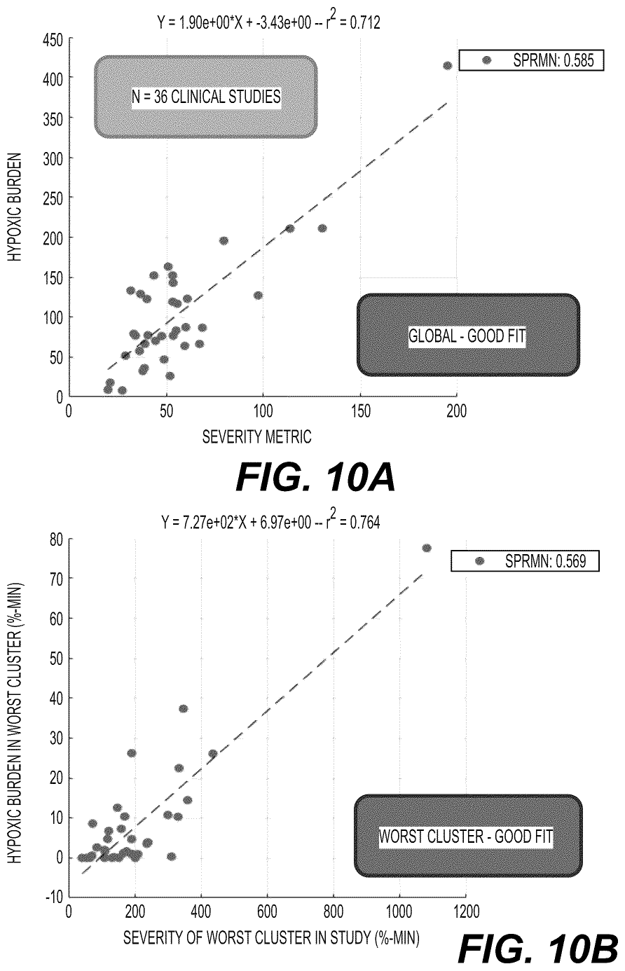

For example, with reference to FIGS. 10A and 10B, real-world severity metric data and hypoxic burden measurements from 36 clinical studies were correlated. As used herein, Hypoxic burden is a compound metric representing the time a user spent in SpO2 desaturation multiplied by the degree of severity of each such desaturation. This is a metric commonly found in sleep literature, and linked to real-world cardiovascular health risks. As shown in FIG. 10A, the global respiratory severity metric (e.g., determined at step 370) was well correlated with Hypoxic burden at approximately 85%. Similarly, as shown in FIG. 10B, the clustering index (e.g., determined at step 360) was well correlated with Hypoxic burden at approximately 85%.

With reference to FIGS. 10A and 10B, severity metrics may be calibrated to a universal scale by using hypoxic burden as a reference point. Such calibration may be performed and set universally for all users 105; performed and set semi-universally based on a user 105's personal characteristics (e.g., age, sex, BMI, weight, fat %, sleep severity diagnosis, etc.); and/or specifically performed and set for a particular user 105 by, for example, assessing recovery time during a sleep study by monitoring Sp02 sensor data or the like. Calibrating with reference to hypoxic burden or SpO2 may be desirable because it is generally known in the art that the higher the hypoxic burden, the greater the risk of severe events and patient harm.

In view of the above, this disclosure contemplates that system 100 may estimate, derive, and/or provide measures of hypoxic burden based off of severity metric data. In turn, this disclosure contemplates that system 100 may estimate, derive, and/or provide measures of accumulated oxygen deficits based off of severity metric data.

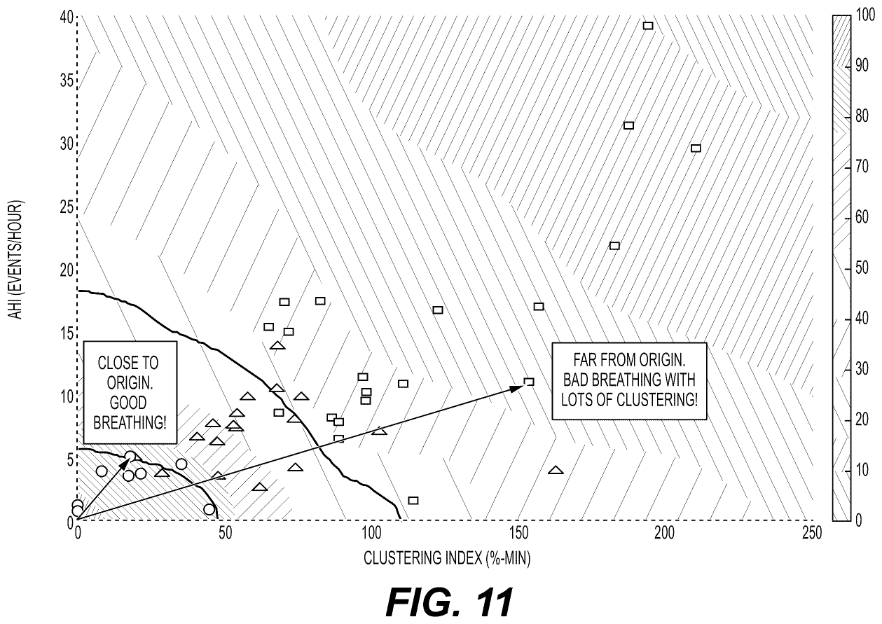

As another example, FIG. 11, based on the clinical sleep studies discussed above and other actual sleep studies, maps “good” sleeps (circles), “fair” sleeps (triangles), and “poor” sleeps (squares) based on data-derived AHI and Clustering index values. As may be observed at the bottom of the chart, AHI alone would consider one “poor” and multiple “fair” sleeps to be “good” (e.g., AHI<5); AHI alone would also consider may “poor” sleeps to be “fair” (e.g., 55<AHI<5). Utilizing the clustering index may help provide a much more accurate assessment.

As may be observed from FIG. 11, severity metric data (e.g. clustering index data) alone may be viewed more accurate than the widely utilized and excepted AHI metric data in assessing sleep severity and sleep problems. It may further be observed that multidimensional assessment—that is based on a severity measure, an AHI measure, and/or others measure may be even more accurate.

Outside of the respiratory severity assessment context, the above described system 100 may be utilized for other sleep-related or non-sleep breathing-assessment purposes.

Although the foregoing embodiments have been described in detail by way of illustration and example for purposes of clarity of understanding, it will be readily apparent to those of ordinary skill in the art in light of the description herein that certain changes and modifications may be made thereto without departing from the spirit or scope of the disclosure. It is also to be understood that the terminology used herein is for the purpose of describing particular aspects only, and is not intended to be limiting, since the scope of the present invention will be limited only by claims.

It is noted that, as used herein, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only,” and the like in connection with the recitation of claim elements, or use of a “negative” limitation. As will be apparent to those of ordinary skill in the art upon reading this disclosure, each of the individual aspects described and illustrated herein has discrete components and features which may be readily separated from or combined with the features of any of the other several aspects without departing from the scope or spirit of the disclosure. Any recited method can be carried out in the order of events recited or in any other order that is logically possible. Accordingly, the preceding merely provides illustrative examples. It will be appreciated that those of ordinary skill in the art will be able to devise various arrangements which, although not explicitly described or shown herein, embody the principles of the disclosure and are included within its spirit and scope.

Furthermore, all examples and conditional language recited herein are principally intended to aid the reader in understanding the principles of the invention and the concepts contributed by the inventors to furthering the art, and are to be construed without limitation to such specifically recited examples and conditions. Moreover, all statements herein reciting principles and aspects of the invention, as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary configurations shown and described herein.

In this specification, various preferred embodiments have been described with reference to the accompanying drawings. It will be apparent, however, that various other modifications and changes may be made thereto and additional embodiments may be implemented without departing from the broader scope of this disclosure. The specification and drawings are accordingly to be regarded in an illustrative rather than restrictive sense.

1. A method for determining at least one respiratory severity metric for an individual's sleep session, the method comprising:
receiving a data signal indicative of respiration of the individual as a function of time;
identifying a plurality of respiratory severity events from the data signal;
calculating respiratory severity values for each of the plurality of identified respiratory severity events;
grouping the plurality of respiratory severity events into a plurality of clusters; and
calculating, for each cluster, an accumulated respiratory severity value as a clustering index.

2. The method of claim 1, further comprising:
designating the highest calculated clustering index as a first of the at least one respiration severity metric.

3. The method of claim 1, further comprising at least one of:
calculating a global measure of accumulated flow reduction during the plurality of identified respiratory events as a first of the at least one respiration severity metric; and
calculating a global measure of all accumulated flow reduction within identified clusters as a first the at least one respiration severity metric.

4. A method for determining at least one respiratory severity metric for an individual's sleep session, the method comprising:
receiving a data signal indicative of respiration of the individual as a function of time from a nasal pressure sensor, an air flow sensor, or a sensor affixed to the individual and configured to detect breathing related bodily movements
identifying a plurality of respiratory severity events from the data signal;
calculating respiratory severity values for each of the plurality of identified respiratory severity events.

5. The method of claim 2, further comprising at least one of:
calculating global measure of accumulated flow reduction during the plurality of identified respiratory events as a second respiration severity metric; and
calculating a global measure of all accumulated flow reduction within identified clusters as a second respiration severity metric.

6. The method of claim 4, further comprising:
calculating global measure of accumulated flow reduction during the plurality of identified respiratory events as a second respiration severity metric;
generating a command to provide wake up signal if the second respiration severity metric exceeds a threshold level.

7. The method of claim 1, further comprising:
receiving a second data signal indicative of a position of the individual as a function of time; and
recording the position of the individual for each cluster.

8. The method of claim 1, wherein the step of receiving the data signal indicative of respiration further comprises:
receiving a signal from a nasal pressure sensor or an air flow sensor.

9. The method of claim 1, wherein the step of receiving the data signal indicative of respiration further comprises:
receiving a signal from a sensor affixed to the individual and configured to detect breathing related bodily movements as a function of time.

10. The method of claim 1, further comprising,
receiving a second data signal indicative of blood oxygenation as a function of time; and
wherein the step of identifying the plurality of respiratory severity events from the data signal further comprises:
identifying each respiratory severity event if and only if the data signal indicates that a clinical definition of apnea or hypopnea has been met, and the second data signal indicates reduced blood oxygenation.

11. The method of claim 1, wherein the step of identifying a plurality of respiratory severity events from the data signal further comprises:
identifying each respiratory severity event if the data signal indicates there has been a predefined change in respiration for at least a set period of time.

12. The method of claim 1, wherein the step off calculating the respiratory severity of each of the plurality of identified respiratory events further comprises, for each identified respiratory event:
calculating an instantaneous flow reduction signal; and
summing the instantaneous flow reduction signal.

13. The method of claim 1, wherein the step of grouping the plurality of respiratory events into clusters further comprises:
including, in a first cluster, a first respiratory event and all subsequent respiratory events, if any, until an air deficit signal indicates a respiratory recovery of the individual.

14. The method of claim 13, further comprising:
calculating the air deficit signal to predict the air deficit of the individual as a function of time.

15. The method of claim 14, wherein the step of calculating the air deficit signal further comprises:
increasing the air deficit signal in accordance with the respiratory severity value of each identified respiratory event; and
decreasing the air deficit signal during periods of time where none of the plurality of identified respiratory events are occurring.

16. The method of claim 15, wherein the step of decreasing the air deficit signal during periods of time where none of the plurality of identified respiratory events are occurring further comprises at least one of:
decaying the air deficit signal linearly based on a set time window;
decaying the air deficit signal linearly based on a set rate of decay;
decaying the air deficit signal non-linearly based on a set time window;
decaying the air deficit signal through application of a fixed length convolution mask;
decaying the air deficit signal linearly based on a variable time window; and
decaying the air deficit signal linearly based on a variable rate of decay.

17. The method of claim 16, wherein the step of decreasing the air deficit signal during periods of time where none of the plurality of identified respiratory events are occurring further comprises:
decaying the air deficit signal at a rate or function based upon the individual's personal characteristics.

18. The method of claim 1, wherein the method is performed in real time during the individual's sleep session, and further comprises:
generating a command to provide wake up signal if the accumulated respiratory severity for at least one cluster exceeds a threshold level.

19. The method of claim 7, wherein the method is performed in real time during the individual's sleep session, and further comprises:
generating a command to provide wake up signal if the accumulated respiratory severity for at least one cluster exceeds a threshold level and the second data signal indicates the individual is in a disfavored position.

20. The method of claim 4, further comprising:
receiving a second data signal indicative of a position of the individual as a function of time;
calculating global measure of accumulated flow reduction during the plurality of identified respiratory events as a second respiration severity metric; and
generating a command to provide wake up signal if the accumulated respiratory severity for at least one cluster exceeds a threshold level and the second data signal indicates the individual is in a disfavored position.
